(19) United States
(12) Patent Application Publication
(10) Pub. No.: US 2024/0293516 A1
(43) Pub. Date: Sep. 5, 2024

(54) FACTOR IX SUBCUTANEOUS ADMINISTRATION WITH ENHANCED SAFETY

(71) Applicant: CSL Behring Lengnau AG, Lengnau BE (CH)

(72) Inventors: Sabine PESTEL, Marburg (DE); Philipp CLAAR, Marburg (DE)

(21) Appl. No.: 18/573,863

(22) PCT Filed: Jun. 28, 2022
(86) PCT No.: PCT/EP2022/067688

§ 371 Date: Dec. 22, 2023

(30) Foreign Application Priority Data
Jul. 1, 2021 (EP) 21183151.6

(51) Int. Cl. A61K 38/36; A61K 9/00; A61P 7/04

(52) U.S. Cl. CPC A61K 38/36; A61K 9/0019; A61P 7/04

(57) ABSTRACT

This invention relates to Factor IX compositions and their uses in therapy by subcutaneous administration. In particular, high-activity Factor IX compositions for subcutaneous administration comprising advantageous Factor IX protein concentrations are provided, which minimize the risk of local tolerability issues, thereby providing enhanced safety.

### TECHNICAL FIELD

This invention relates to Factor IX compositions and their uses in therapy by subcutaneous administration. In particular, high-activity Factor IX compositions for subcutaneous administration comprising advantageous Factor IX protein concentrations are provided, which minimise the risk of local tolerability issues, thereby providing enhanced safety.

### BACKGROUND

Human coagulation Factor IX (FIX) plays a key role in the formation of blood clots. FIX has been used in the prophylaxis and treatment of bleeding disorders, such as hemophilia B.

Existing therapies administer FIX via intravenous infusion. Subcutaneous administration of FIX has the potential to provide a number of advantages, such as the ability of patients to self-administer FIX more easily. A major challenge in the subcutaneous delivery of FIX is that sufficiently high FIX activity doses must be delivered for effective prophylaxis or treatment. However, there are practical limits on the volume that can be administered per subcutaneous administration site. Additionally, it is desirable to use as few subcutaneous administration sites as possible, while achieving the necessary total FIX activity dose. A further challenge lies in the considerable loss in FIX bioavailability when it is administered subcutaneously as compared to the intravenous route. Administering FIX at a high concentration has the potential to address these challenges. It is unclear, however, whether the resulting high concentration of FIX is well tolerated at the subcutaneous administration site. For example, FIX is a bioactive molecule that is involved in the coagulation cascade. Blood clotting is a self-perpetuating process that is desirable at sites of injury, but high local concentrations/doses of FIX could inadvertently result in excessive local clotting (e.g., thrombus formation), thereby leading to adverse effects at the subcutaneous administration site.

The present invention is based on the surprising realisation that an important factor in determining local tolerability of subcutaneously administered FIX is the FIX protein concentration in the administered composition, rather than the administered FIX activity concentration/dose. Hence, the risk of local tolerability issues caused by the subcutaneous administration of the necessary high FIX activity doses could be minimised by administering high-activity FIX protein at a lower FIX protein concentration, even where the total administered FIX activity dose remains unchanged or is even, advantageously, higher.

### DISCLOSURE OF THE INVENTION

The present invention provides advantageous protein concentrations of Factor IX which minimise the risk of local tolerability issues following subcutaneous administration. The invention is suitable for use with high-activity Factor IX variants, such as the R338L (‘Padua’) variant, as described in ref. 1. Other high-activity Factor IX variants for use in the invention are known to the skilled person and are described herein. The use of a high-activity variant enables the administration of high Factor IX activity levels at low protein concentrations per subcutaneous injection site, as the inventors have discovered that local tolerability is primarily determined by Factor IX protein concentration. Thus, by administering high-activity variants of FIX, high Factor IX activity levels can be administered subcutaneously while maintaining a low, safe protein concentration. Lower injection volumes per subcutaneous injection site, and/or administration at fewer injection sites, can therefore be used, which improves patient comfort, compliance and safety. A Factor IX variant for use with the invention may further be linked to a half-life enhancer (e.g., albumin) which provides the Factor IX variant with a longer functional half-life in vivo. Compositions for subcutaneous administration in accordance with the invention are particularly useful in the prevention or treatment of bleeding disorders such as hemophilia B.

In one aspect, the invention therefore provides a high-activity Factor IX (FIX) variant polypeptide for use in therapy of a human subject, wherein the FIX variant polypeptide is to be administered subcutaneously at a FIX protein concentration of no more than 20 mg/mL and in a total volume of no more than 5 mL per administration site.

Provided herein is also a composition comprising a high-activity Factor IX (FIX) variant polypeptide for use in therapy of a human subject, wherein the composition comprises a FIX protein concentration of no more than 20 mg/mL, and no more than 5 mL of the composition is to be administered subcutaneously to the subject.

The invention therefore provides a high-activity Factor IX (FIX) variant polypeptide, and compositions comprising the same, for use in therapy of a human subject, wherein the FIX variant polypeptide is to be administered subcutaneously at a FIX protein concentration that is safe, for example as assessed by the absence of thrombus formation at the subcutaneous administration site.

The terms “high-activity FIX variant polypeptide”, “high-activity FIX variant”, “variant”, “FIX polypeptide” and so on are used interchangeably herein and all refer to a high-activity FIX variant polypeptide unless expressly stated otherwise.

Unless indicated otherwise, “FIX protein” (e.g., in “FIX protein concentration”) herein refers to the weight of the FIX portion (e.g., as defined in SEQ ID NO: 8) in the protein, i.e. excluding the weight of any additional portions such as fusion partners (e.g., albumin).

Unless specifically stated otherwise the term “administration” or “administering” or “administered” refers to subcutaneous administration.

Corresponding methods for treatment, and uses of a high-activity FIX variant polypeptide in the manufacture of a medicament, are also provided.

Administration of “no more than” a certain protein concentration of FIX polypeptide in a total volume of “no more than” a certain volume of FIX polypeptide requires that some FIX polypeptide is administered, and therefore excludes embodiments wherein no FIX polypeptide is administered.

In certain embodiments, the FIX polypeptide is administered at a FIX protein concentration of no more than 19 mg/mL, no more than 18 mg/mL, no more than 17 mg/mL, no more than 16 mg/mL, or no more than 15.8 mg/mL per administration site. In one embodiment, the FIX polypeptide is administered at a FIX protein concentration of no more than 19 mg/mL. In another embodiment, the FIX polypeptide is administered at a FIX protein concentration of no more than 18 mg/mL. In a further embodiment, the FIX polypeptide is administered at a FIX protein concentration of no more than 17 mg/mL. In a yet further embodiment, the FIX polypeptide is administered at a FIX protein concentration of no more than 16 mg/mL. Thus, in an exemplary embodiment, the FIX polypeptide is administered at a FIX protein concentration of no more than 15.8 mg/mL.

In certain embodiments, the FIX polypeptide is administered at a FIX protein concentration of between and 2 mg/mL and 20 mg/mL. For example, the FIX polypeptide may be administered at a FIX protein concentration of between 2 mg/ml and 16 mg/mL, between 5 mg/ml and 16 mg/mL, between 7 mg/mL and 16 mg/mL, or between 9 mg/mL and 16 mg/mL. In an exemplary embodiment, the FIX polypeptide is administered at a FIX protein concentration of between 4.6 mg/mL and 15.8 mg/mL.

Where the FIX polypeptide is an albumin fusion protein (e.g., SEQ ID NO: 10), the FIX protein concentration may also be expressed with reference to the weight of the entire FIX albumin fusion protein. Accordingly, the invention also provides a high-activity Factor IX (FIX) variant polypeptide for use in therapy of a human subject, wherein the FIX variant polypeptide is to be administered subcutaneously at a protein concentration of no more than 48 mg/ml (e.g., no more than 47 mg/mL, no more than 45 mg/mL, no more than 43 mg/mL, no more than 41 mg/mL, no more than 39 mg/mL, no more than 37 mg/mL, preferably no more than 37 mg/mL) and in a total volume of no more than 5 mL per administration site (preferably no more than 1 mL, e.g., no more than 0.5 mL), wherein the protein concentration is the amount in mg of the entire protein (e.g., SEQ ID NO: 10) divided by volume in mL. Thus in an exemplary embodiment the invention provides a high-activity Factor IX (FIX) variant polypeptide for use in therapy of a human subject, wherein the FIX variant polypeptide is to be administered subcutaneously at a protein concentration of no more than 37 mg/mL and in a total volume of no more than 1 mL, wherein the protein concentration is the amount in mg of the entire protein (e.g., SEQ ID NO: 10) divided by volume in mL.

In certain embodiments, the FIX polypeptide is administered at a FIX activity concentration of more than 2000 IU/mL, such as at least 3000 IU/mL, for example at least or about 3500 IU/mL, at least or about 4200 IU/mL, at least or about 5000 IU/mL, at least or about 10000 IU/mL, or at least or about 15000 IU/mL, or even at least or about 16000 IU/mL or 16500 IU/mL. For example, the FIX polypeptide may be administered at a FIX activity concentration of at least 3000 IU/mL. In an exemplary embodiment, the FIX polypeptide may be administered at a FIX activity concentration of between 3000 IU/mL and 16500 IU/mL.

In other embodiments, the FIX polypeptide is administered at a FIX activity concentration of no more than 5000 IU/mL. The FIX polypeptide may be administered at a FIX activity concentration of no more than 10000 IU/mL. The FIX polypeptide may be administered at a FIX activity concentration of no more than 15000 IU/mL. The FIX polypeptide may be administered at a FIX activity concentration of no more than 16000 IU/mL. The FIX polypeptide may be administered at a FIX activity concentration of no more than 16500 IU/mL. In other embodiments, the FIX polypeptide is administered at a FIX activity concentration of about 4200 IU/mL. In another exemplary embodiment, the FIX polypeptide is administered at a FIX activity concentration of about 5000 IU/mL, e.g. between 3000 IU/mL and 5000 IU/mL.

In certain embodiments, the FIX polypeptide is administered at a total FIX activity dose (per spot) of at least or about 5000 IU per administration site. In other embodiments, the FIX polypeptide is administered at a total FIX activity dose of at least or about 5000 IU, at least or about 7500 IU, at least or about 10000 IU, at least or about 12500 IU, or at least or about 15000 IU. For example, the FIX polypeptide may be administered at a total FIX activity dose 10 of between 5000 IU and 10000 IU per administration site. The FIX polypeptide may be administered at a total FIX activity dose of between 5500 IU and 7500 IU per administration site. In an exemplary embodiment, the FIX polypeptide is administered at a total FIX activity dose of about 5000 IU per administration site. In an exemplary embodiment, the FIX polypeptide is administered at a total FIX activity dose of about 5500 IU per administration site. In a further exemplary embodiment, the FIX polypeptide is administered at a total FIX activity dose (per spot) of about 7500 IU per administration site.

The FIX dose in IU/kg to be administered to a subject can be determined by a skilled person using methods known in the art, e.g. taking into account the FIX activity rise that is desired (% of normal or IU/dl).

In certain embodiments, the FIX polypeptide is administered in a total volume of no more than 4.5 mL, no more than 4 mL, no more than 3.5 mL, no more than 3 mL, no more than 2.5 mL, no more than 2 mL, no more than 1.5 mL, or no more than 1 mL per administration site. In one embodiment, the FIX polypeptide is administered in a total volume of no more than about 4 mL (e.g. 4.2 mL) per administration site. In another embodiment, the FIX polypeptide is administered in a total volume of no more than about 2.5 mL per administration site. In another embodiment, the FIX polypeptide is administered in a total volume of no more than 1.6 mL per administration site. In another embodiment, the FIX polypeptide is administered in a total volume of no more than about 1.3 mL per administration site. In a further embodiment, the FIX polypeptide is administered in a total volume of no more than 1.0 mL per administration site. For example, the FIX polypeptide may be administered in a total volume of between 1.3 mL and 4.2 mL per administration site. In certain embodiments, the FIX polypeptide is administered in a total volume of between 0.1 mL and 2.5 mL per administration site, for 35 example between 0.25 mL and 2.5 mL per administration site, or between 0.5 mL and 2.5 mL per administration site. In other embodiments, the FIX polypeptide is administered in a total volume of between 0.1 mL and 1.6 mL per administration site, for example between 0.1 mL and 1.0 mL per administration site, or between 0.5 mL and 1.0 mL per administration site. In preferred embodiments, the FIX polypeptide is administered in a total volume of no more than 2 mL per administration site, and even more preferred in a total volume of no more than 1 mL per administration site. For example, the FIX polypeptide may be administered in a total volume of no more than 1 mL per administration site using an autoinjector. In yet further embodiments, the FIX polypeptide is administered in a total volume of around 1 mL per administration site, e.g. between 0.5 mL and 1.5 mL, or between 0.8 mL and 1.2 mL, optionally using an autoinjector.

In any of these embodiments, the FIX polypeptide is typically administered as a bolus injection. In certain embodiments, the FIX variant polypeptide has a specific molar activity that is at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, or at least 10 times higher than the specific molar activity of a FIX polypeptide containing wild-type FIX (e.g. a polypeptide containing a wild-type FIX as defined in SEQ ID NO: 1, for example a FIX polypeptide as defined in SEQ ID NO: 9). For example, the FIX variant polypeptide may have a specific molar activity of at least 3 times higher than the specific molar activity of a FIX polypeptide containing wild-type FIX. In another embodiment, the FIX variant polypeptide has a specific molar activity that is at least 5 times higher than the specific molar activity of a FIX polypeptide containing wild-type FIX. In a further embodiment, the FIX variant polypeptide has a specific molar activity that is at least 7 times higher than the specific molar activity of a FIX polypeptide containing wild-type FIX.

For example, in one embodiment, the FIX variant polypeptide has a specific molar activity of up to 25 times higher than the specific molar activity of a FIX polypeptide containing wild-type FIX, or up to 20 times higher than the specific molar activity of a FIX polypeptide containing wild-type FIX, e.g., between 3 to 25, between 3 to 20, between 3 to 15, or between 3 to 10 times higher than the specific molar activity of a FIX polypeptide containing wild-type FIX. In one embodiment, the FIX variant polypeptide has a specific molar activity of between 3 to 8 times higher than the specific molar activity of a FIX polypeptide containing wild-type FIX, e.g., the FIX variant polypeptide has a specific molar activity of between 5 to 8 times higher than the specific molar activity of a FIX polypeptide containing wild-type FIX.

Where the FIX variant polypeptide and wild-type FIX have the same molecular weight, the ratios provided above also apply when expressed based on the activity per mg of polypeptide. In certain embodiments, the FIX polypeptide is a variant that comprises the amino acid leucine (L) at a position corresponding to position 338 of wild-type Factor IX. The numbering refers to the amino acid positions in the wild-type Factor IX as identified in SEQ ID NO: 1. Other FIX variants can be used in the invention, including those provided in Table 1.

It will be understood by those skilled in the art that the term “amino acid” in the context of a polypeptide is used interchangeably with “amino acid residue”.

In certain embodiments, the high-activity FIX variant polypeptide (e.g. comprising the amino acid leucine (L) at a position corresponding to position 338 of wild-type Factor IX) comprises an amino acid sequence having at least 70% sequence identity to SEQ ID NO: 1. In a specific embodiment, the FIX polypeptide comprises an amino acid sequence having at least 75%, at least 80%, at least 85%, at least 90%, at least 95%, at least 96%, at least 97%, at least 98%, or at least 99% sequence identity to SEQ ID NO: 1. In a particular embodiment, the FIX variant polypeptide comprises an amino acid sequence having at least 95% sequence identity to SEQ ID NO: 1. In any of these embodiments, the Factor IX variant polypeptide is biologically active, i.e. it is capable of activating Factor X (i.e. generating Factor Xa, with a higher activity than wild-type FIX).

For example, in one embodiment, the high-activity FIX variant polypeptide (e.g. comprising the amino acid leucine (L) at a position corresponding to position 338 of wild-type Factor IX) comprises an amino acid sequence having at least 80% sequence identity to SEQ ID NO: 1.

In another embodiment, the high-activity FIX variant polypeptide (e.g. comprising the amino acid leucine (L) at a position corresponding to position 338 of wild-type Factor IX) comprises an amino acid sequence having at least 90% sequence identity to SEQ ID NO: 1.

For example, the high-activity FIX variant polypeptide (e.g. comprising the amino acid leucine (L) at a position corresponding to position 338 of wild-type Factor IX) comprises an amino acid sequence having at least 95% sequence identity to SEQ ID NO: 1.

In one embodiment, the high-activity FIX variant polypeptide does not comprise the amino acid arginine (R) at a position corresponding to position 5 of wild-type FIX, e.g., the high-activity FIX variant polypeptide does not comprise the K5R mutation.

In a particular embodiment, all residues in the Factor IX variant polypeptide other than at position 338 are wild-type, i.e. there is a 100% sequence identity with SEQ ID NO: 1 excluding the amino acid at position 338.

Thus, in one embodiment the high-activity FIX variant polypeptide comprises the amino acid sequence as defined in SEQ ID NO: 8. The Factor IX variant polypeptide may also comprise a biologically active fragment of SEQ ID NO: 8 (i.e. a fragment that has procoagulant activity, for example corresponding to activated Factor IX) and comprising the amino acid at position 338 of SEQ ID NO: 8.

In any of these embodiments, the high-activity Factor IX variant polypeptide is biologically active, i.e. it is capable of activating Factor X (i.e. generating Factor Xa, with a higher activity than wild-type FIX).

In any of these embodiments, the FIX polypeptide may be connected (or “linked”) to a half-life enhancing portion. In particular embodiments, the half-life enhancing portion is selected from the group consisting of albumin including variants and derivatives thereof, polypeptides of the albumin family including variants and derivatives thereof (e.g. recombinant human albumin), immunoglobulins without antigen binding domain (e.g. the Fc portion, such as lgG Fc), polyethylene glycol, a C-terminal peptide of human chorionic gonadotropin (CTP), or an unstructured recombinant polypeptide (e.g. XTEN). Alternatively, a FIX polypeptide molecule for use in the invention may consist of the Factor IX polypeptide provided herein (e.g. as defined in SEQ ID NO: 8), i.e., without any additional portion(s), such as half-life enhancing portion(s).

The “volume of distribution” (Vss or Vz, mL/kg) may be taken into account when identifying FIX polypeptides that are particularly suitable for use in the invention. In any of the embodiments described herein, the FIX polypeptide may have a volume of distribution following subcutaneous administration (i.e., in the extravascular space) to a human subject that is comparable to (e.g., +40% or less, such as +30%, +20%, or +10%) or that is the same as that of an albumin-fused FIX (e.g., Idelvion® as defined in SEQ ID NO: 9, or the ‘Padua’ mutant as defined in SEQ ID NO: 10). In certain embodiments, the FIX polypeptide may have a volume of distribution following subcutaneous administration to a human subject that is not greater than that of an albumin-fused FIX (as defined above). The volume of distribution can be determined using methods known to the person skilled in the art.

In certain embodiments, the Factor IX variant polypeptide is connected to a half-life enhancing portion wherein the half-life enhancing portion is albumin including variants and derivatives thereof, or a polypeptide of the albumin family including variants and derivatives thereof, optionally wherein the FIX variant polypeptide is as defined in SEQ ID NO: 10. The Factor IX polypeptide may be linked in any of these ways directly or via a linker. The linker may be a cleavable linker, for example a proteolytically cleavable linker. Alternatively, a non-cleavable linker may be used. In a preferred embodiment, the half-life enhancing portion is an albumin, which is linked to the FIX portion via a cleavable linker. In a specific embodiment, the FIX polypeptide is as defined in SEQ ID NO: 10.

In certain embodiments, the FIX polypeptide is for use in the treatment or prophylaxis of bleeding in a subject with a bleeding disorder, in particular a subject with hemophilia B.

The invention also provides a pharmaceutical composition suitable for subcutaneous administration, wherein the pharmaceutical composition comprises a high-activity Factor IX (FIX) variant polypeptide at a FIX protein concentration of no more than 20 mg/ml and in a total volume of no more than 5 mL per unit dose.

The FIX protein concentration and total volume can be provided according to any one of the embodiments described herein. The FIX polypeptide in the composition can also be provided according to any one of the embodiments described herein. Other features of the composition, e.g., the FIX protein dose, FIX activity concentration, or FIX activity dose can also be provided according to any one of the embodiments described herein.

In another aspect, the invention provides an injection device, e.g. an autoinjector device, suitable for subcutaneous administration and containing a pharmaceutical composition in accordance with the invention. In one embodiment, the injection device holds a volume of up to or around 1.0 mL.

### Factor IX Polypeptide

Any suitable high-activity Factor IX variant polypeptide can be used in the invention. A suitable Factor IX polypeptide is one that when administered to a subject subcutaneously according to the invention provides the desired safety and efficacy. Typically, a desired safety and efficacy is wherein plasma FIX trough levels prevent bleeding and no local tolerability issues (e.g. no more than minimal thrombus formation) are observed at the subcutaneous injection site(s).

The Factor IX polypeptide is typically a recombinant polypeptide. The Factor IX polypeptide is typically comprised of naturally occurring amino acid. However, one or more non-naturally occurring amino acids can also be present. The Factor IX polypeptide for use in the invention may be provided in purified form. The Factor IX polypeptide for use in the invention may be provided in isolated form. The Factor IX polypeptide may be post-translationally modified.

The Factor IX polypeptide is biologically active, i.e. it is capable of activating Factor X (i.e. generating Factor Xa, with a higher specific activity than wild-type FIX).

A particularly suitable Factor IX variant polypeptide is derived from a polypeptide sequence of wild-type Factor IX (SEQ ID NO: 1). The variant differs at one or more amino acid positions from the corresponding positions in the wild-type Factor IX, i.e. the variant has one or more amino acid substitutions relative to the corresponding positions in the wild-type Factor IX.

The Factor IX variant polypeptide for use in the invention has an increased FIX specific activity relative to a wild-type Factor IX polypeptide, e.g. relative to the Factor IX polypeptide from which the variant is derived. Such a Factor IX polypeptide is also referred to herein as a ‘high-activity’ Factor IX polypeptide, or a high-activity Factor IX variant polypeptide. Other terms are used in the art synonymously, e.g. ‘hyperactive’ FIX variants.

The variant has the biological function of a Factor IX, i.e. the variant is able to generate Factor Xa, optionally after the Factor IX variant polypeptide has been converted to its active form (Factor IXa) by excision of the activation peptide. The variant is able to generate Factor Xa with a higher activity than wild-type FIX. Activation cleavage of Factor IX can be achieved in vitro e.g. by Factor Xla or Factor Vlla/TF. Suitable in vitro assays to measure Factor IX activity are known to the person skilled in the art (e.g. one-stage clotting assay such as an aPTT assay, chromogenic assay, etc.). An in vitro aPTT-based one stage clotting assay is a preferred assay for determining Factor IX activity, typically as described in the Examples.

An exemplary high-activity Factor IX variant polypeptide comprises leucine (L) at a position corresponding to position 338 of wild-type Factor IX, which typically has an arginine (R) at that position (“R338L”). One such exemplary polypeptide is the ‘Padua’ mutant, described in ref. 1, see SEQ ID NO: 8. The specific activity of the ‘Padua’ mutant is typically at least around 5-8 fold higher compared to wild-type Factor IX.

Other exemplary high-activity Factor IX variants are E410H, E410K, R338V, and R338L+E410K, and those described in WO 2020/187969 A1 (ref. 2), e.g. comprising the amino acid H at a position corresponding to position 410 of wild-type Factor IX, and comprising an amino acid other than R at a position corresponding to position 338 of wild-type Factor IX, for example comprising an amino acid selected from the group consisting of V, T and W at a position corresponding to position 338 of wild-type Factor IX, e.g. R338V+E410H, R338T+E410H, R338W+E410H, and R338L+E410H.

Further exemplary high-activity Factor IX variant polypeptides include those listed in Table 1 below.

TABLE 1

| | Exemplary fold | |
|---|---|---|
| | change in FIX | |
| Amino acid | activity relative | |
| substitution(s) | to FIX-WT | Reference(s) |
| E410H | 4.6 | ref. 3 |
| G4Y | 1.2 | ref. 4 |
| V10K | 1.6 | ref. 4, ref. 5 |
| V86A | 1.1 | ref. 6 |
| K265T | 1.9 | ref. 7, ref. 8 |
| E277A | 1.3 | ref. 6 |
| N346A | 1.2 | ref. 9 |
| S377W | 1.4 | ref. 4 |
| R338L + S377W | 12 | ref. 4 |
| V10K + R338L + S337W | 19 | ref. 4 |
| R318Y + R338E + T343R | 17 | ref. 10 |
| V86A + E277A + R338L | 22 | ref. 11 |
| G4Y + V86A + R338L + S337W | 15 | ref. 4 |

The numbering in Table 1 refers to the positions in the mature FIX protein without propeptide sequence (SEQ ID NO: 1). The activity was determined with a one-stage clotting assay.

The skilled person is able to identify and verify these and other high-activity Factor IX variant polypeptides, by determining the specific (molar) activity of a Factor IX polypeptide using methods known in the art and comparing that activity with wild-type Factor IX. Further explanations on determining Factor IX specific activity are provided below (see the section entitled “Factor IX activity”) and in the Examples.

The Factor IX variant polypeptide can be derived from a Factor IX polypeptide sequence of any mammalian species. In a particular embodiment, the Factor IX variant polypeptide is derived from a Factor IX polypeptide sequence of human origin. Gene ID: 2158 (https://www.ncbi.nlm.nih.gov/gene/2158), GenBank Accession Nos. NM_000133.3 (https://www.ncbi.nlm.nih.gov/nuccore/NM_000133.3), NP_000124.1 (https://www.ncbi.nlm.nih.gov/protein/NP_000124.1?report=genpept), and UniProt entry P00740 (https://www.uniprot.org/uniprot/P00740) provide examples of the amino acid and/or nucleotide sequences of wild-type human Factor IX.

The Factor IX variant polypeptide according to the invention may be derived from mature (i.e. excluding signal peptide and propeptide) wild-type Factor IX, for example of human origin, the amino acid sequence of which is shown in SEQ ID NO: 1. That polypeptide sequence is ‘isoform 1’ of human Factor IX.

The polypeptide of SEQ ID NO: 1 has the amino acid R at position 338 (references to amino acids herein use the single-letter codes as widely known in the art; for example, “R” stands for arginine, etc.). Position 338 in SEQ ID NO: 1 is in the Peptidase S1 domain. Position 338 is indicated in bold and underlined below. The 35-aa activation peptide which is excised to form FIXa (activated Factor IX) is underlined.

(SEQ ID NO: 1)
YNSGKLEEFVQGNLERECMEEKCSFEEAREVFENTERTTEFWKQYVDGD
QCESNPCLNGGSCKDDINSYECWCPFGFEGKNCELDVTCNIKNGRCEQF
CKNSADNKVVCSCTEGYRLAENQKSCEPAVPFPCGRVSVSQTSKLTRAE
TVFPDVDYVNSTEAETILDNITQSTQSFNDFTRVVGGEDAKPGQFPWQV
VLNGKVDAFCGGSIVNEKWIVTAAHCVETGVKITVVAGEHNIEETEHTE
QKRNVIRIIPHHNYNAAINKYNHDIALLELDEPLVLNSYVTPICIADKE
YTNIFLKFGSGYVSGWGRVFHKGRSALVLQYLRVPLVDRATCLRSTKFT
IYNNMFCAGFHEGGRDSCQGDSGGPHVTEVEGTSELTGIISWGEECAMK
GKYGIYTKVSRYVNWIKEKTKLT

An exemplary polynucleotide coding sequence for the polypeptide of SEQ ID NO: 1 is shown in SEQ ID NO: 2.

The term “derived from a polypeptide sequence of wild-type Factor IX” (or similar wording) means that the Factor IX variant polypeptide has some degree of sequence identity with wild-type Factor IX polypeptide when the two sequences are aligned. For example, the Factor IX variant polypeptide may have at least 70% etc. sequence identity to SEQ ID NO: 1, as described above. The Factor IX variant polypeptide is biologically active, i.e. it is capable of activating Factor X (i.e. generating Factor Xa, with a higher specific activity than wild-type FIX).

A percentage sequence identity between two amino acid sequences means that, when aligned, that percentage of amino acids is the same in comparing the two sequences. The percentage sequence identity is calculated as the percentage of identical amino acids within the aligned sequences. A sequence that “has” (or “having”) x % sequence identity to another sequence means that the sequence is x % identical to that other sequence.

In embodiments in which the Factor IX polypeptide is linked with a half-life enhancing portion (e.g. albumin), optionally via a cleavable linker, i.e. a fusion protein, when determining the sequence identity with SEQ ID NO: 1 only the Factor IX portion of the molecule is considered for the purposes of calculating the sequence identity, i.e. excluding any linker and excluding the half-life enhancing portion of the molecule. This applies also when, e.g., the linker is derived from a Factor IX sequence, i.e. the linker is excluded for the purposes of calculating the sequence identity with SEQ ID NO: 1.

Similarly, where the Factor IX polypeptide corresponds to one or more fragments of the full-length Factor IX polypeptide (e.g. it is an activated form of Factor IX), when determining the sequence identity with SEQ ID NO: 1 any portions that are present in SEQ ID NO: 1 but missing in the Factor IX polypeptide (e.g. the activation peptide) are excluded for the purposes of calculating the sequence identity.

The term “wild-type Factor IX” refers to a Factor IX polypeptide sequence that occurs naturally and has a FIX activity that is typical of natural FIX such as that found in standard human plasma. The sequence has not been artificially modified relative to the sequence of the naturally occurring polypeptide sequence. This means that none of the amino acids in the naturally occurring polypeptide sequence has been substituted with a different amino acid. SEQ ID NO: 1 is an example of a wild-type polypeptide sequence, but functional fragments, truncations, etc. are also encompassed by the term, as exemplified below. For example, the term includes polypeptides with a modified N-terminal or C-terminal end including terminal amino acid deletions or additions, as long as those polypeptides substantially retain the activity of wild-type Factor IX. The term also includes any natural polymorphic variant of Factor IX. For example, a common natural polymorphic variant which occurs with a frequency of 33% is a Factor IX polypeptide presenting an alanine (A) in a position corresponding to position T148 in SEQ ID NO: 1. This T148A polymorphic variant is shown in SEQ ID NO: 7. All references to SEQ ID NO: 1 herein may therefore also refer to SEQ ID NO: 7.

The Factor IX variant polypeptide may also be derived from a wild-type Factor IX that includes the signal and/or the propeptide, as shown in SEQ ID NO: 3. SEQ ID NO: 3 includes both the signal peptide (aa 1-28) and the propeptide (aa 29-46). The polypeptide of SEQ ID NO: 3 is known in the art as the precursor of human Factor IX, or as the prepropeptide Factor IX. Factor IX with propeptide but lacking the signal peptide is also known as a propeptide Factor IX. An exemplary polynucleotide coding sequence encoding the polypeptide of SEQ ID NO: 3 is shown in SEQ ID NO: 4.

The Factor IX variant polypeptide may also be derived from one or more functional fragments of wild-type Factor IX, for example it may be derived from activated Factor IX which contains two fragments of Factor IX (it is missing the intervening ‘activation peptide’ that is present in SEQ ID NO: 1). SEQ ID NOs 5 and 6 show the light chain and heavy chain, respectively, of human activated Factor IX, which are held together by a disulphide bridge. Another example is isoform 2 of human Factor IX, which lacks the 38-aa stretch at positions 47-84 of SEQ ID NO: 1.

Alternatively, the Factor IX variant polypeptide may be derived from a truncation or a fusion of wild-type Factor IX.

The Factor IX variant polypeptide therefore may take various different forms, as long as it maintains the biological function of Factor IX as described above (i.e. it is a functional Factor IX variant polypeptide, with a higher specific activity than wild-type FIX). Accordingly, the Factor IX variant polypeptide of the invention may be a variant of a wild-type prepropeptide Factor IX, propeptide Factor IX, mature Factor IX, activated Factor IX, or their functional fragments, truncations, fusions, isoforms, polymorphic variants, etc. All of these forms of Factor IX are collectively referred to herein, unless indicated otherwise, as ‘Factor IX’.

References to amino acid positions made herein are relative to the numbering in SEQ ID NO: 1, i.e. the amino acid positions are those corresponding to that position in SEQ ID NO: 1. This means that, for example, if a Factor IX variant polypeptide is based on SEQ ID NO: 1 but additionally includes the propeptide and signal peptide of Factor IX (which together are 46 amino acids long, and are missing from SEQ ID NO: 1), then e.g. “a Factor IX variant polypeptide comprising the amino acid L at a position corresponding to position 338 of wild-type Factor IX” means that the Factor IX variant polypeptide comprises L at position 384 of the variant polypeptide 338 plus 46). Similarly, if the Factor IX variant polypeptide is based on an activated version of Factor IX (which lacks the 35-aa activation peptide of SEQ ID NO: 1), then e.g. “a Factor IX variant polypeptide comprising the amino acid L at a position corresponding to position 338 of wild-type Factor IX” means that the Factor IX variant polypeptide comprises L at position 303 of the variant polypeptide (338 minus 35), which corresponds to position 158 of the heavy chain of activated Factor IX. The skilled person is able to determine the relevant positions in a Factor IX variant polypeptide by comparing the polypeptide sequence of the variant with the polypeptide sequence of SEQ ID NO: 1 and identifying the aligning portion(s).

The Factor IX variant polypeptide may be provided as an “isolated” or as a “purified” polypeptide. This term may refer to a polypeptide produced by expression of an isolated nucleic acid molecule of the invention. Alternatively, this term may refer to a protein which has been sufficiently separated from other proteins with which it would naturally be associated (e.g., so as to exist in “substantially pure” form). “Isolated” is not meant to exclude artificial or synthetic mixtures with other compounds or materials, or the presence of impurities that do not interfere with the fundamental activity, and that may be present, for example, due to incomplete purification, or the addition of stabilizers.

The term “substantially pure” refers to a preparation comprising at least 75% by weight of Factor IX variant polypeptide, particularly at least 80% by weight, at least 85% by weight, at least 90% by weight, at least 95% by weight, or at least 96%, 97%, 98%, or 99% by weight, e.g. 90-99% or more by weight of Factor IX variant polypeptide. Purity may be measured by methods appropriate for the compound of interest (e.g. chromatographic methods, polyacrylamide gel electrophoresis, HPLC analysis, and the like).

A Factor IX variant polypeptide (or a pharmaceutical composition comprising the same) according to the invention may be therapeutic when administered subcutaneously to a subject (e.g. a human), e.g. a subject with Factor IX deficiency such as hemophilia B, i.e. a prophylactic or therapeutic effect can be observed. This means that the plasma levels of Factor IX activity can be increased, at least temporarily. Such a prophylactic or therapeutic effect can be determined for example by measuring the plasma Factor IX activity in the subject after subcutaneous administration, and comparing it to the plasma Factor IX activity in that subject before the administration. An increase in Factor IX activity after subcutaneous administration indicates a prophylactic or therapeutic effect. A prophylactic or therapeutic effect is also achieved where the Factor IX activity after subcutaneous administration is sufficient to prevent, reduce or inhibit bleeding. This includes the situation where an increase in FIX levels cannot be measured in the plasma but the functional consequences of increased FIX activity can be observed in the form of prevented, reduced or inhibited bleeding. The Factor IX activity after subcutaneous administration preferably results in troughs of at least 15-40%, or may even be outside of the pathological range.

Factor IX activity can be measured using any Factor IX activity assay known to the skilled person, for example using a clotting activity assay, such as an aPTT assay (a decrease in aPTT value indicates increased Factor IX activity). In a preferred embodiment therefore Factor IX activity is determined using a clotting activity assay, e.g. an in vitro aPTT-based one stage clotting assay, typically as described in the Examples.

A Factor IX variant polypeptide for use in the invention is preferably non-immunogenic when administered subcutaneously to a subject, typically a human subject. This means that after subcutaneous administration of the polypeptide to the subject, the subject does not exhibit a measurable immune response (e.g. neutralising antibodies) against the variant polypeptide beyond that observed against the corresponding wild-type polypeptide. However, any such immune response can be avoided or treated if necessary, e.g. with corticosteroids. Tests for evaluating immunogenicity are known in the art, e.g. Example 11 of reference 12.

A Factor IX variant polypeptide for use in the invention is also preferably non-thrombogenic when administered subcutaneously to a subject, typically a human subject. This means that after subcutaneous administration of the polypeptide to the subject, the subject does not experience any, or only minimal, local (e.g. at the injection site) or systemic thrombosis.

### Factor IX Activity

Factor IX activity is generally referred to as the specific activity. The specific activity is defined as the activity per mg of the entire polypeptide of interest. This means that in a fusion protein, e.g., of FIX and albumin, the specific activity of said fusion protein is based on (calculated with) the weight of the total mass of the fusion protein.

The molar specific activity is defined as the activity per mole (or e.g. nmole) of the entire polypeptide of interest. Calculation of the molar specific activity allows a direct comparison of the activity of different polypeptides. The molar specific activity is not affected by the different molecular weights or optical densities of the different polypeptides. The molar specific activity may be calculated as exemplified in table 2 of reference 13.

Factor IX activity may be determined using any suitable assay. Various Factor IX activity assays are well known to the skilled person in the art, e.g. one-stage clotting assay, aPTT assay, and chromogenic assay. An activated partial thromboplastin time (aPTT) assay is a well-known Factor IX assay. In a preferred embodiment therefore Factor IX activity is determined using an in vitro aPTT-based one stage clotting assay. Such an exemplary assay is described in the Examples below. It is commercially available (e.g., Pathromtin® SL, Siemens Healthcare). Incubation of test sample plasma (e.g. Factor IX depleted plasma containing an amount of sample, e.g. from a subject, a cell culture supernatant, or a purified Factor IX polypeptide) with the optimal quantity of phospholipids and a surface activator leads to activation of factors of the intrinsic coagulation system. The addition of calcium ions triggers the coagulation process; the time to formation of a fibrin clot is measured. An internal substandard calibrated against the WHO International FIX concentrate Standard can be used as a reference. One International Unit (IU) of factor IX is equivalent to the amount of factor IX in one mL of normal human plasma (1 U/mL or 1%). However, other known Factor IX activity assays may also be used to determine the activity of a Factor IX polypeptide.

An “increase” in specific activity relative to a control occurs when such an increase is observed in at least one Factor IX activity assay, e.g. a reduction in aPTT value when Factor IX activity is measured using an in vitro aPTT-based one stage clotting assay, for example as described in the Examples.

### Factor IX Protein

FIX protein amount is determined via Optical Density (OD) measurement, using methods well known to the skilled person in the art. An exemplary method is described in the Examples, wherein protein content by OD was determined using the Little Lunatic (Unchained Labs, Pleasanton, CA 94566) instrument.

Unless indicated otherwise, “FIX protein” (e.g., in “FIX protein concentration”) herein refers to the weight of the FIX portion (e.g., as defined in SEQ ID NO: 8) in the protein, i.e. excluding the weight of any additional portions such as fusion partners (e.g., albumin). To correct the protein mass for the albumin weight, the following assumptions were taken: molecular mass was calculated from the FASTA-sequence of the molecules (FIX=52 kDa, albumin=69 kDa, albumin-fused FIX=121 kDa). On this basis, the mass of the FIX portion of the albumin-fusion protein is 43% of the total protein mass.

### Factor IX Antigen

Factor IX antigen may be determined using any suitable assay. An exemplary ELISA-based assay is described in the Examples. FIX antigen concentration in a test sample is calculated using a standard curve with standard human plasma as reference.

### Therapy

In one aspect the invention relates to therapeutic uses of a high-activity Factor IX variant, by subcutaneous administration.

The use may be the treatment or prophylaxis of bleeding in a subject with a bleeding disorder. The bleeding disorder may be any disorder which requires pro-coagulant (e.g., to prevent, reduce or inhibit bleeding). An exemplary bleeding disorder is hemophilia, particularly hemophilia B.

The invention therefore provides a method for the treatment or prophylaxis of a disorder in a subject in need thereof comprising subcutaneously administering a prophylactically or therapeutically effective amount of a Factor IX polypeptide to the subject. By a “therapeutically effective amount” it is meant that the administration of that amount of Factor IX polypeptide to a subject, either in a single dose or as part of a series, is effective for treatment or prophylaxis. Such methods have efficacy in the prophylaxis or treatment of disorders where a pro-coagulant activity is needed (e.g., to prevent, reduce or inhibit bleeding) and include, without limitation, hemophilia, particularly hemophilia B. Thus, in one embodiment, the invention provides a method for the prophylaxis of bleeding in a subject in need thereof comprising subcutaneously administering a prophylactically or therapeutically effective amount of a Factor IX polypeptide to the subject. In another embodiment, the invention provides a method for the treatment of bleeding in a subject in need thereof comprising subcutaneously administering a prophylactically or therapeutically effective amount of a Factor IX polypeptide to the subject. The invention also provides a Factor IX polypeptide for use in therapy, i.e. for use as a medicament, wherein the FIX variant polypeptide is administered subcutaneously as described herein. The term “therapy” includes treatment and prophylaxis. Thus, in one embodiment, the invention provides a Factor IX polypeptide for use in the prophylaxis of bleeding, wherein the FIX variant polypeptide is administered subcutaneously as described herein. In another embodiment, the invention provides a Factor IX polypeptide for use in the treatment of bleeding, wherein the FIX variant polypeptide is administered subcutaneously as described herein.

Also provided is the use of a Factor IX polypeptide for the manufacture of a medicament for treating a subject, wherein the FIX variant polypeptide is to be administered subcutaneously to the subject as described herein. Thus, in one embodiment, the invention provides Factor IX polypeptide for the manufacture of a medicament for the prophylaxis of bleeding, wherein the FIX variant polypeptide is to be administered subcutaneously to the subject as described herein. In another embodiment, the invention provides Factor IX polypeptide for the manufacture of a medicament for the treatment of bleeding, wherein the FIX variant polypeptide is to be administered subcutaneously to the subject as described herein.

In any of these embodiments, the treatment or prophylaxis of bleeding is in a subject with a bleeding disorder.

The treatment or prophylaxis may include on-demand control of bleeding episodes, perioperative management of bleeding, and/or routine prophylaxis to prevent or reduce the frequency of bleeding episodes. For example, treatment may include on-demand control of bleeding episodes or perioperative management of bleeding. Prophylaxis may include prevention of bleeding episodes or reducing the frequency of bleeding episodes.

The subject is typically a human. The subject may be an adult or a child. The subject may have a basal (without prophylaxis or treatment) plasma Factor IX activity of 40% or less, 30% or less, 20% or less, 10% or less, 5% or less, 4% or less, 3% or less, 2% or less, between 1-5%, or 1% or less, compared to the plasma Factor IX activity of a healthy subject. In a particular embodiment, the subject is a paediatric subject (a child), e.g., 18 years or younger. In one embodiment, the subject is not eligible to receive FIX gene therapy.

In one embodiment, the FIX polypeptide is administered in a composition that does not contain anti-thrombotic substances (e.g., heparin).

Bleeding disorders include hemophilia (hemophilia A, hemophilia B, hemophilia A and B patients with inhibitory antibodies; in particular hemophilia B), deficiencies in at least one coagulation factor (e.g., Factors VII, IX, X, XI, V, XII, II, and/or von Willebrand factor; in particular Factor IX), combined FV/FVIII deficiency, vitamin K epoxide reductase CI deficiency, gamma-carboxylase deficiency; bleeding associated with trauma, injury, thrombosis, thrombocytopenia, stroke, coagulopathy (hypocoagulability), disseminated intravascular coagulation (DIC); over-anticoagulation associated with heparin, low molecular weight heparin, pentasaccharide, warfarin, small molecule antithrombotics (i.e. FXa inhibitors); and platelet disorders such as, Bernard Soulier syndrome, Glanzman thromblastemia, and storage pool deficiency.

In a preferred embodiment, the method or use described above is for the treatment or prophylaxis of bleeding in a subject with hemophilia B, which is also known in the art as congenital factor IX deficiency.

The terms “treatment”, “therapy” and “treating” may include prophylaxis, unless indicated otherwise. The terms “treatment”, “therapy” and “treating” also include on-demand treatment. A disorder is treated or prevented if administration of a Factor IX polypeptide to a subject (e.g. a human with Factor IX deficiency such as hemophilia B) results in a therapeutic or prophylactic effect. This means that the plasma level of Factor IX activity in the subject is increased following treatment, at least temporarily, when measured with at least one Factor IX assay. The Factor IX activity can be determined using an in vitro aPTT-based one stage clotting assay (e.g. as described in the Examples). The increase may be clinically relevant, e.g. a reduction in the frequency or intensity of bleeding events.

One way of expressing Factor IX activity in plasma is as a percentage relative to normal human plasma. Another way of expressing Factor IX activity in plasma is in International Units (IU) relative to an International Standard for Factor IX in plasma. One IU of Factor IX activity in plasma is equivalent to that quantity of Factor IX in one mL of normal human plasma.

One way of checking efficacy of prophylaxis or treatment is by measuring the plasma Factor IX activity in the subject after prophylaxis or treatment, and comparing it to the plasma Factor IX activity in that subject before prophylaxis or treatment. An increase in Factor IX activity after prophylaxis or treatment (e.g. from <1%, or 1%-5%, or 5-40% of normal human plasma to e.g., 15%, 20%, >25%, >30%, >35%, >40%, >50%, or >60% peak levels of normal human plasma, e.g. from <5% to >5% such as to 5-40%) indicates a prophylactic or therapeutic effect. Factor IX levels of 5-10% of normal human serum have been targeted in clinical trials for achieving bleeding control while on prophylaxis.

A prophylactic or therapeutic effect is also achieved where the Factor IX activity after prophylaxis or treatment is sufficient to prevent, reduce or inhibit bleeding.

The Factor IX activity after prophylaxis or treatment may results in troughs of at least 15-40%, or may even be outside of the pathological range (e.g. >40% peak levels of normal human serum).

Factor IX activity can be measured using any Factor IX activity assay known to the skilled person, for example using an aPTT assay (a decrease in aPTT value indicates increased Factor IX activity). In a preferred embodiment therefore Factor IX activity is determined using an in vitro aPTT-based one stage clotting assay, e.g. as described in the Examples.

A Factor IX variant polypeptide for use in the invention may have a higher specific molar activity when administered in vivo to a subject than the corresponding wild-type Factor IX polypeptide. For example, the % increase in plasma Factor IX activity (e.g. measured using an in vitro aPTT-based one stage clotting assay) may be higher with a Factor IX variant polypeptide as described herein as compared with using the same molar amount of the corresponding wild-type Factor IX polypeptide. Another way of describing this is that the aPTT time in a serum sample after administering a Factor IX variant polypeptide as described herein is shorter as compared with the same molar amount of the corresponding wild-type Factor IX polypeptide.

### Preparing a Factor IX Variant Polypeptide

A Factor IX variant polypeptide for use in the invention can be made using standard techniques well known to the skilled person in the art. For example, the cDNA sequence of a wild-type Factor IX (e.g. SEQ ID NO: 2) may be modified using standard mutagenesis techniques (e.g. site-directed mutagenesis) so that it encodes the desired Factor IX variant polypeptide, e.g. encoding the amino acid L at a position corresponding to position 338 of wild-type Factor IX (which encodes R at that position). An N-terminal leader peptide for the purposes of recombinant protein production can be used, based on the natural Factor IX leader peptide (as shown in SEQ ID NO: 3) or alternatives known to the skilled person in the art. The cDNA sequence may be inserted into a suitable expression plasmid to express the recombinant Factor IX variant polypeptide. This is typically performed using mammalian cells (e.g. HEK for transient expression or a CHO cell line for stable expression), although other types of cells that can produce glycosylated and correctly folded proteins can also be used. The recombinant Factor IX variant polypeptide may subsequently be purified, for example using anion exchange chromatography.

A Factor IX variant polypeptide for use in the invention can also be provided as part of a fusion with another moiety, e.g. with an albumin (for example attached via a cleavable linker).

The Factor IX variant polypeptide may be combined with other agents and/or with a pharmaceutically acceptable carrier. A composition comprising the Factor IX variant polypeptide may also be provided in lyophilised form.

### Fusions and Conjugates

The Factor IX polypeptide may be provided in fusion with, or it may be conjugated to, one or more additional portions. The one or more additional portions are typically different from Factor IX, i.e. they do not have the biological function of Factor IX as defined above (they do not have the ability to generate Factor Xa). This means that fragments of Factor IX, e.g. linkers comprising a fragment of a Factor IX-derived polypeptide sequence, but which do not on their own have the function of Factor IX, may be such “one or more additional portions”, i.e. they are not part of the Factor IX portion but they may be part of the molecule that comprises the Factor IX portion.

### Half-Life Enhancing Portion and Linker

In an exemplary embodiment, the Factor IX portion is linked to a half-life enhancing portion. The half-life enhancing portion may comprise one or more polypeptides (half-life enhancing polypeptides, HLEPs). In one embodiment, the HLEP is albumin, e.g. recombinant human albumin. In another embodiment, the HLEP is a fragment of an antibody (immunoglobulin), such as the Fc fragment, e.g. IgG Fc, such as IgG1 Fc. Alternatively, the HLEP may be a C-terminal peptide of human chorionic gonadotropin (CTP). The HLEP may also be an unstructured recombinant polypeptide (e.g. XTEN). Such molecules are also referred to in the art as fusion polypeptides.

The Factor IX portion may be linked to the HLEP via a cleavable linker. Typically, the cleavable linker is cleavable by the same protease that activates Factor IX. Such cleavable linkers therefore provide a high molar specific activity of the fusion polypeptide. Suitable cleavable linkers are taught, for example, in reference 13.

The Factor IX polypeptide may also be PEGylated, i.e. one or more polyethylene glycol moieties are conjugated to the Factor IX polypeptide, using methods known in the art.

A Factor IX polypeptide for use in the invention may comprise one half-life enhancing portion, or more than one half-life enhancing portions. The wording “a half-life enhancing portion” therefore covers one or more half-life enhancing portions. The half-life enhancing portions may be of the same type. The half-life enhancing portions may be of different types. For example, the Factor IX polypeptide may be linked to XTEN (e.g. XTEN72) and additionally to an Fc domain (e.g. human lgG1 Fc).

Preferably, the half-life enhancing portion is capable of extending the half-life of the Factor IX polypeptide in vivo (in plasma) by at least about 25% as compared to the non-fused Factor IX polypeptide. Preferably, the half-life enhancing portion is capable of extending the half-life of the Factor IX polypeptide in vivo (in plasma) by at least about 50%, and more preferably by more than 100%. The in vivo half-life is generally determined as the terminal half-life or the ß-half-life.

### Albumin

As used herein, “albumin” refers collectively to an albumin polypeptide or amino acid sequence, or an albumin fragment, variant or analog having one or more functional activities (biological activities) of albumin. In particular, “albumin” may refer to human albumin (HA) or a fragment thereof, especially the mature form of human albumin as shown in SEQ ID NO: 12 herein. The albumin may also be derived from other species, in particular other vertebrates.

The albumin portion of the fusion polypeptide may comprise the full length of the HA sequence as described in SEQ ID NO: 12, or it may include one or more fragments thereof that are capable of stabilizing or prolonging the therapeutic activity of the Factor IX variant polypeptide. Such fragments may be of 10 or more amino acids in length or may include about 15, 20, 25, 30, 50, or more contiguous amino acids from the HA sequence or may include part or all of the specific domains of HA. These and other suitable albumin portions (including variants) are described in reference 13.

Structurally related family members of the albumin family may also be used as HLEPs. For example, alpha-fetopolypeptide (AFP, reference 12) is a member of the albumin family and may also be used to enhance the half-life of a Factor IX variant polypeptide. Such half-life enhancing polypeptides are described in reference 13. Another option is afamin (AFM, reference 14) or vitamin D binding polypeptide (DBP, reference 15). Fragments of these polypeptides may also be used.

In embodiments that use albumin HLEPs, the albumin is typically provided as a genetic fusion with the Factor IX portion. This means that a single cDNA molecule encodes the Factor IX portion and the albumin portion, optionally with an intervening sequence encoding a linker, such as a cleavable linker.

### Immunoglobulin

An immunoglobulin (lg) or a fragment thereof may also be used as a HLEP. An example of a suitable immunoglobulin is IgG, or an IgG-fragment, such as an Fc region. The Fc region may be an Fc domain (e.g., two polypeptide chains each of which comprises the hinge region (or part of the hinge region), the CH2 region and the CH3 region). Thus, a Factor IX polypeptide may be fused to an Fc domain, directly or via a linker. In embodiments that use a linker, the linker may be cleavable.

Monomers, dimers and hybrids are all encompassed. For example, the Factor IX polypeptide may be a heterodimer comprising two polypeptide chains, wherein the first chain comprises a Factor IX portion linked to the hinge region (or part of the hinge region), the CH2 region and the CH3 region of an immunoglobulin (e.g. lgG1), and the second chain comprises the hinge region (or part of the hinge region), the CH2 region and the CH3 region of an immunoglobulin (e.g. lgG1).

In another embodiment, the Factor IX polypeptide is a homodimer comprising two polypeptide chains, wherein each chain comprises a Factor IX portion linked to the hinge region (or part of the hinge region), the CH2 region and the CH3 region of an immunoglobulin (e.g. lgG1).

In a further embodiment, the Factor IX polypeptide is a monomer comprising a Factor IX portion linked to the hinge region (or part of the hinge region), the CH2 region and the CH3 region of an immunoglobulin (e.g. lgG1).

Other examples of suitable Factor IX IgG Fc fusion molecule configurations are found, e.g., in reference 16.

An exemplary Fc polypeptide (derived from the human lgG1 Fc domain) is shown in SEQ ID NO: 13. Another exemplary Fc polypeptide (derived from the human lgG1 Fc domain) is shown in SEQ ID NO: 14.

In any of these embodiments, the Factor IX portion may be linked directly or via a linker to the Fc portion. In embodiments that use a linker, the linker may be cleavable or non-cleavable. In particular embodiments, the linker is cleavable. An exemplary cleavable linker is shown in SEQ ID NO: 11.

An exemplary Fc portion is the Fc portion of Eftrenonacog alfa (Alprolix®). See also references 17, 18 or 19.

### C-Terminal Peptide of Human Chorionic Gonadotropin (CTP)

Another exemplary half-life enhancing portion is a C-terminal peptide of human chorionic gonadotropin (CTP). CTP is based on a natural peptide of 31 amino acids length, the C-terminal peptide of the beta chain of human chorionic gonadotropin (hCG).

One or more units of CTP can be fused to a Factor IX portion. The one or more units of CTP can be fused to the N-terminus and/or to the C-terminus of Factor IX, preferably to the C-terminus.

In one embodiment, the Factor IX polypeptide is a CTP-modified Factor IX comprising a Factor IX polypeptide as described herein linked with three to five CTPs, optionally wherein the CTPs are attached to the C-terminus of the Factor IX polypeptide. In a specific embodiment, three tandem units of CTP are attached the Factor IX polypeptide, optionally at the C-terminus of the Factor IX polypeptide.

In any of these embodiments, at least one of the CTP may be attached to the Factor IX portion via a linker. The linker may be a peptide bond. The linker may be cleavable.

In an exemplary embodiment, the CTP sequence comprises SEQ ID NO: 15. In another exemplary embodiment, the CTP sequence comprises SEQ ID NO: 16. In another exemplary embodiment, the CTP sequence comprises SEQ ID NO: 17.

Other suitable CTP sequences and related methods are known to the skilled person in the art, e.g. see references 20, 21 or 22.

### Unstructured Recombinant Polypeptide

Another exemplary half-life enhancing portion is an unstructured recombinant polypeptide. An example of such an unstructured recombinant polypeptide is XTEN, see e.g. reference 23.

In one embodiment, the Factor IX polypeptide therefore is a Factor IX polypeptide fused with at least one XTEN. XTEN may be fused to the Factor IX portion by insertion into the Factor IX polypeptide sequence while maintaining the biological activity of Factor IX. For example, the XTEN may be inserted between two neighbouring amino acids in the activation peptide of Factor IX at a position that does not prevent cleavage of the activation peptide during coagulation when XTEN is inserted. Alternatively, XTEN may be fused to the C-terminus and/or N-terminus of the Factor IX, preferably the C-terminus. XTEN may be fused to the C-terminus and/or N-terminus (preferably C-terminus) of the Factor IX via a linker, e.g. a cleavable linker. The linker may be cleavable by thrombin.

A preferred XTEN is XTEN72. An exemplary XTEN72 sequence is shown in SEQ ID NO: 18. An alternative XTEN sequence is shown in SEQ ID NO: 19. Other suitable sequences and methods are disclosed in e.g. references 24, 25 or 26.

In a specific embodiment, the Factor IX polypeptide comprises XTEN72 linked to the activation peptide of Factor IX and wherein the Factor IX portion is furthermore linked to a human IgG1 Fc domain at the C-terminus of the Factor IX portion.

### PEGylation

Another exemplary half-life enhancing portion is polyethylene glycol (PEG). GlycoPEGylation is within the scope of the term “PEGylation” as used herein. For example, a ca. 40 kDa PEG portion may be covalently attached to the Factor IX polypeptide, for example via a specific N-linked glycan within the activation peptide.

An example of a glycoPEG moiety is the glycoPEG moiety is nonacog beta pegol (Refixia®) (see also reference 27), in which an average of one non-reducing end of a glycan at N157 or N167 of Factor IX (numbering according to SEQ ID NO: 1) is attached to neuraminic acid conjugated to two PEG polymers (total average molecular weight of the polymers is ca. 42 kDa) via the amino group.

PEGylation of Factor IX polypeptide is also taught, for example, in references 28, 29 and 30.

### Linker

A Factor IX polypeptide comprising a half-life enhancing portion may employ a cleavable linker, in particular a proteolytically cleavable linker. The linker is generally positioned between the Factor IX polypeptide portion and a half-life enhancing portion. The linker may liberate the Factor IX portion upon cleavage of the linker by a protease of the coagulation cascade, e.g. a protease that is also capable of converting Factor IX to its activated form, e.g., FXIa or VIIa/tissue factor (TF). Cleavable linkers are particularly useful when the HLEP is albumin. Although it is desirable to have an enhanced Factor IX in vivo half-life, it is desirable to limit the half-life of the Factor IX once it has been activated, to reduce the risk of a prothrombotic effect, especially with a hyperactive Factor IX variant polypeptide. In some embodiments therefore, a cleavable linker links the Factor IX variant polypeptide to a half-life enhancing portion, thereby providing a Factor IX variant polypeptide with a longer half-life relative to a non-fusion polypeptide. However, once bleeding occurs and the coagulation cascade has been initiated, a protease of the coagulation cascade activates the Factor IX variant polypeptide which has increased specific activity relative to e.g. the corresponding wild-type Factor IX. At the same time, the linker is cleaved and the activated Factor IX variant polypeptide is liberated from the half-life enhancing portion, thereby reducing the risk of a prothrombotic effect due to any prolonged increased Factor IX activity.

The linker may be a fragment of Factor IX, preferably a fragment that is involved in Factor IX activation. For example, the linker may comprise such a fragment of a Factor IX sequence, extended by an N-terminal residue, such as a proline residue. An exemplary cleavable linker is shown in SEQ ID NO: 11. Other cleavable linkers are described in reference 13.

A Factor IX variant polypeptide linked to a half-life enhancing portion via an intervening cleavable linker may have at least 25% higher molar specific activity compared to the corresponding molecule with a non-cleavable linker (e.g. GGGGGGV, SEQ ID NO: 20), when measured in at least one coagulation-related assay, examples of which are known to the skilled person in the art, e.g. an aPTT one-stage assay, for example as described in the Examples. Preferably, a Factor IX variant polypeptide linked to a half-life enhancing portion via an intervening cleavable linker has at least 50%, more preferably at least 100% increased molar specific activity compared to the corresponding molecule without cleavable linker.

### Pharmaceutical Compositions

Also provided herein is a pharmaceutical composition comprising a high-activity Factor IX variant polypeptide. The composition is for administration to a subject, such as an animal, typically a human subject. The composition is suitable for subcutaneous administration, optionally after reconstitution or dilution.

Thus, in one embodiment provided herein is a pharmaceutical composition suitable for subcutaneous administration, wherein the pharmaceutical composition comprises a high-activity Factor IX (FIX) variant polypeptide at a FIX protein concentration of no more than 20 mg/mL and in a total volume of no more than 5 mL per unit dose.

In specific embodiments, the FIX protein concentration per unit dose of pharmaceutical composition (i.e. per single injection) is no more than 19 mg/mL, no more than 18 mg/mL, no more than 17 mg/mL, no more than 16 mg/mL, or no more than 15.8 mg/ml per administration site.

In any of these embodiments, the total volume per unit dose of pharmaceutical composition (i.e. per single injection) is no more than 4.5 mL, no more than 4 mL, no more than 3.5 mL, no more than 3 mL, no more than 2.5 mL, no more than 2 mL, no more than 1.5 mL, or no more than 1 mL per administration site. For example, the total volume is no more than 3 mL. Another example is a total volume of no more than 2 mL. Preferably, the total volume is no more than 1 mL, e.g. 0.1-1 mL.

The pharmaceutical composition is pharmaceutically acceptable and typically includes a suitable carrier. A thorough discussion of pharmaceutically acceptable carriers is available in reference 31. The composition is preferably sterile, pyrogen- and/or preservative-free.

Thus, the Factor IX polypeptide may be provided in buffered liquid form, e.g. in a citrate buffer, optionally containing a stabiliser and/or a bulking agent. An exemplary pharmaceutical composition for use in the invention comprises a Factor IX polypeptide (a high-activity Factor IX variant polypeptide), tri-sodium citrate dihydrate, polysorbate 80, mannitol, sucrose, hydrochloric acid, and sterile water. In an exemplary formulation, the components are 25 mM tri-sodium citrate dihydrate, 0.006%-0.024% polysorbate 80, 18-29 g/L mannitol, 7-12 g/L sucrose, hydrochloric acid for adjusting the pH to 6.6-7.2 (e.g. pH 6.8), and sterile water. In a preferred embodiment, the formulation is tri-sodium-citrate-2*H2O 30 mmol/L, D-mannitol 35.5 g/L, sucrose 14.0 g/L, polysorbate 80 0.00030 mL/L, pH 7.0.

Alternatively, the Factor IX polypeptide in the composition is lyophilized but is reconstituted with liquid diluent prior to subcutaneous administration, e.g. with sterile water for injection. Typical excipients in a composition comprising lyophilized Factor IX polypeptide include tri-sodium citrate dihydrate, polysorbate 80, mannitol, sucrose, and/or hydrochloric acid.

Compositions may be prophylactic (to prevent bleeding) or therapeutic (to treat bleeding).

The invention includes the following embodiments:
1. A high-activity Factor IX (FIX) variant polypeptide for use in therapy of a human subject, wherein the FIX variant polypeptide is to be administered subcutaneously at a FIX protein concentration of no more than 20 mg/mL and in a total volume of no more than 5 mL per administration site.
2. The FIX variant polypeptide for use of embodiment 1, wherein the FIX variant polypeptide is to be administered at a FIX protein concentration of no more than 19 mg/mL, no more than 18 mg/mL, no more than 17 mg/mL, no more than 16 mg/mL, or no more than 15.8 mg/ml per administration site.
3. The FIX variant polypeptide for use of embodiment 1 or embodiment 2, wherein the FIX variant polypeptide is to be administered in a total volume of no more than 4.5 mL, no more than 4 mL, no more than 3.5 mL, no more than 3 mL, no more than 2.5 mL, no more than 2 mL, no more than 1.5 mL, no more than 1 mL, or no more than 0.5 mL per administration site.
4. The FIX variant polypeptide for use of any one of the preceding embodiments, wherein the FIX variant polypeptide is to be administered in a total volume of no more than 1 mL per administration site.
5. The FIX variant polypeptide for use of any one of the preceding embodiments, wherein the FIX variant polypeptide is to be administered at a FIX protein concentration of no more than 15.8 mg/mL and in a total volume of no more than 1 mL per administration site.
6. The FIX variant polypeptide for use of any one of the preceding embodiments, wherein the FIX variant polypeptide is to be administered at a FIX protein concentration of no more than 15.8 mg/mL and in a total volume of no more than 0.5 mL per administration site.
7. The FIX variant polypeptide for use of any one of the preceding embodiments, wherein the FIX variant polypeptide has a specific molar activity that is at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, or at least 10 times higher than the specific molar activity of a FIX polypeptide containing wild-type FIX.
8. The FIX variant polypeptide for use of embodiment 7, wherein the FIX variant polypeptide has a specific molar activity that is at least 3 times higher than the specific molar activity of a FIX polypeptide containing wild-type FIX, for example wherein the FIX variant polypeptide has a specific molar activity that is between 3 to 8 times higher than the specific molar activity of a FIX polypeptide containing wild-type FIX.
9. The FIX variant polypeptide for use of any one of the preceding embodiments, wherein the FIX variant polypeptide comprises the amino acid L at a position corresponding to position 338 of wild-type Factor IX as defined in SEQ ID NO: 1.
10. The FIX variant polypeptide for use of any one of the preceding embodiments, wherein the Factor IX variant polypeptide comprises an amino acid sequence having at least 70% sequence identity to SEQ ID NO: 1, for example at least 80%, at least 90%, or at least 95%.
11. The FIX variant polypeptide for use of any one of the preceding embodiments, wherein the FIX variant polypeptide comprises an amino acid sequence as defined in SEQ ID NO: 8.
12. The FIX variant polypeptide for use of any one of the preceding embodiments, wherein the Factor IX variant polypeptide is connected to a half-life enhancing portion, in particular wherein the half-life enhancing portion is albumin including variants and derivatives thereof, or a polypeptide of the albumin family including variants and derivatives thereof, optionally wherein the FIX variant polypeptide is as defined in SEQ ID NO: 10.
13. The FIX variant polypeptide for use of any one of the preceding embodiments, for use in treatment or prophylaxis of bleeding in a human subject with hemophilia B.
14. A pharmaceutical composition suitable for subcutaneous administration, wherein the pharmaceutical composition comprises a high-activity Factor IX (FIX) variant polypeptide at a FIX protein concentration of no more than 20 mg/mL and in a total volume of no more than 5 mL per unit dose.
15. The pharmaceutical composition of embodiment 14, wherein the high-activity Factor IX (FIX) variant polypeptide is as defined in any one of embodiments 7-12.

### General

The practice of the present invention will employ, unless otherwise indicated, conventional methods of chemistry, biochemistry, molecular biology, immunology and pharmacology, within the skill of the art. Such techniques are explained fully in the literature.

The term “comprising” encompasses “including” as well as “consisting” e.g. a composition “comprising” X may consist exclusively of X or may include something additional e.g. X+Y.

The term “about” in relation to a numerical value x is optional and means, for example, x+10%.

The word “substantially” does not exclude “completely” e.g. a composition which is “substantially free” from Y may be completely free from Y. Where necessary, the word “substantially” may be omitted from the definition of the invention.

The following examples are provided to illustrate various embodiments of the present invention. The examples are illustrative and are not intended to limit the invention in any way.

### BRIEF DESCRIPTION OF THE DRAWINGS

FIG. 1: Plasma exposure of FIX using an ELISA method in the pig model at 0-96 hours after subcutaneous administration of fusion protein containing WT or R338L FIX.

FIG. 2: Images of injection sites in rabbits after subcutaneous administration of 0.9 mg/ml (700 IU/mL, 1.6 mL) R338L FIX (A), 2.5 mg/ml (2000 IU/mL, 1.0 mL) R338L FIX (B), or 20.1 mg/ml (2051 IU/mL, 1 mL) WT FIX (C).

### EXAMPLES

The Examples described below inter alia demonstrate that, surprisingly, local tolerability of subcutaneously administered FIX depends on the FIX protein concentration, rather than, e.g., FIX activity or protein dose. Accordingly, a FIX protein concentration threshold is proposed, below which FIX can be administered subcutaneously with a lower risk of local tolerability issues. The threshold is based on the pig model, which has skin that is physiologically similar to humans. The invention is therefore suitable for use with high-activity FIX variants, such as the FIX ‘Padua’ (R338L) mutant, thereby allowing the subcutaneous administration of a sufficiently high (i.e. prophylactically or therapeutically effective) FIX activity dose at a sufficiently low FIX protein concentration so as to minimise the risk of local tolerability issues.

### Example 1

### Subcutaneously Administered Factor IX Wild-Type and High-Activity Variant in the Pig Model

Pigs were used as an in vivo model for the subcutaneous administration of Factor IX (FIX) to human subjects. Pigs are suitable models for human subcutaneous therapy because porcine skin is very similar to human skin, for example in terms of the skin's general structure, hydrodynamic pressure, thickness, hair follicle content, pigmentation, collagen and lipid composition. See, e.g., ref. 32.

Nine groups of pigs (Sus scrofa domestica, wild type) were tested, as indicated in Table 2 below. “WT”=wild-type Factor IX (with albumin fusion); “HA”=high-activity Factor IX (with albumin fusion). The protein dose/concentration (e.g., “FIX protein concentration”) is based on the weight of the Factor IX portion of the protein, excluding the weight of the albumin portion in the fusion protein. The “Specific activity [IU/mg]” is based on the clotting activity per mg of the entire polypeptide (including the albumin portion).

TABLE 2

| | | Specific | Activity | | Protein | | Activity | | Protein | | | | | | | |
|---|---|---|---|---|---|---|---|---|---|---|---|---|---|---|---|---|
| | | activity | dose | | dose | | dose per | | dose per | | | | Activity | | FIX protein | |
| Group | Protein | [IU/mg] | per kg | | per kg | | spot | | spot | | Volume | | concentration | | concentration | |
| 1 | WT | 60 | 27 | IU/kg | 190 | μg/kg | 615 | IU | 4.4 | mg | 1.67 | mL | 369 | IU/mL | 2.6 | mg/mL |
| 2 | HA | 220 | 25 | IU/kg | 49 | μg/kg | 582 | IU | 1.1 | mg | 0.17 | mL | 3456 | IU/mL | 6.8 | mg/mL |
| 3 | HA | 220 | 248 | IU/kg | 485 | μg/kg | 5575 | IU | 10.9 | mg | 1.61 | mL | 3456 | IU/mL | 6.8 | mg/mL |
| 4 | WT | 64 | 248 | IU/kg | 1664 | μg/kg | 7524 | IU | 50.6 | mg | 10.73 | mL | 701 | IU/mL | 4.7 | mg/mL |
| 5 | WT | 62 | 238 | IU/kg | 1656 | μg/kg | 7205 | IU | 50.2 | mg | 2.39 | mL | 3019 | IU/mL | 21.0 | mg/mL |
| 6 | HA | 404 | 253 | IU/kg | 269 | μg/kg | 7500 | IU | 8.0 | mg | 1.59 | mL | 4727 | IU/mL | 5.0 | mg/mL |
| 7 | HA | 436 | 226 | IU/kg | 223 | μg/kg | 7500 | IU | 7.4 | mg | 0.70 | mL | 9514 | IU/mL | 9.4 | mg/mL |
| 8 | HA | 441 | 254 | IU/kg | 248 | μg/kg | 7500 | IU | 7.3 | mg | 0.52 | mL | 16239 | IU/mL | 15.8 | mg/mL |
| 9 | HA | 432 | 249 | IU/kg | 248 | μg/kg | 7500 | IU | 7.5 | mg | 1.78 | mL | 4585 | IU/mL | 4.6 | mg/mL |

Factor IX albumin fusion protein containing either wild-type Factor IX (“WT”, SEQ ID NO: 9) or a high-activity Factor IX (R338L variant, SEQ ID NO: 10) was administered subcutaneously as indicated above. The group 1 formulation contained FIX as defined in SEQ ID NO: 9 (1000 IU vial, dissolved with water for injection leading to the buffer components tri-sodium-citrate-2*H2O 25 mmol/L, D-mannitol 29.2 g/L, sucrose 11.5 g/L, polysorbate 80 0.00025 mL/L, pH 6.8). The group 4 formulation also contained FIX as defined in SEQ ID NO: 9 (3500 IU vial, dissolved with water for injection leading to the buffer components tri-sodium-citrate-2*H2O 30 mmol/L, D-mannitol 35.5 g/L, sucrose 14.0 g/L, polysorbate 80 0.00030 mL/L, pH 7.0). The group 2, 3 and 6-9 formulations containing FIX as defined in SEQ ID NO: 10, and the group 5 formulation containing FIX as defined in SEQ ID NO: 9, were formulated in 20 mM Hepes, 100 mM isotonic saline, 10 mmol CaCl₂), pH 6.2). Previous studies supported that the buffers did not affect local tolerability.

The subcutaneous FIX injections were performed under ketamine, xylazine and azaperone anaesthesia, with one subcutaneous injection spot per animal for FIX administration. Each animal received the respective FIX dose into one flank and a control saline dose (of the same dose volume) was administered into the contralateral flank. For groups 6-9 an additional buffer control was added on the same side as saline.

Blood samples (1 part sodium citrate 3.13%+9 parts blood) were collected by puncturing the ear up to 96 h post-administration (predose, 5 min, 15 min, 1 h, 2 h, 4 h, 6 h, 24 h, 30 h, 48 h, 72 h, 96 h p.a.). Plasma samples were stored at approximately−70° C. until analysis. Factor IX antigen (FIX:Ag) plasma levels were measured with the anti-human FIX ELISA (Cedarlane, Ontario, Canada), as described below.

The injection site was monitored at predose, and at every subsequent blood sampling time point. Any potential abnormalities at the injection site were macroscopically inspected and documented in case of any changes. After 96 h p.a., the animals were sacrificed, and a necropsy was performed. Again, any abnormalities at the injection site were macroscopically inspected and documented.

Skin and muscle tissue samples were taken in deep anaesthesia and fixed in 10% formalin solution for microscopic histological evaluation. Histological evaluation included the evaluation of the incidence and severity of: inflammatory cell infiltrate in the dermis/epidermis, subcutis and muscle, serocellular crust, haemorrhage, and thrombus.

### Results

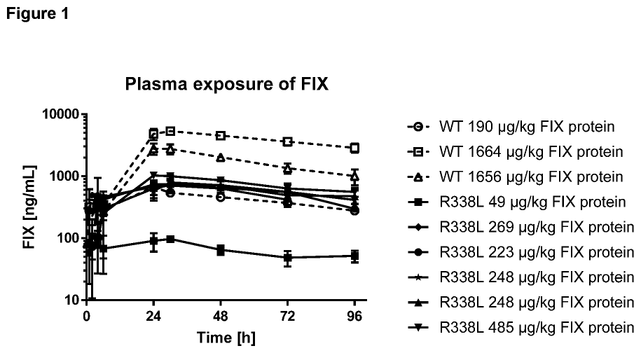

Subcutaneously administered FIX entered the circulation and was detected in plasma. At Cmax (at 24 h-30 h p.a.), FIX plasma levels increased with FIX protein dose, and were comparable between WT and HA at similar FIX protein doses, i.e. at about 200-250 μg/kg, with regard to AUCo-inf (40,000-50,000 h*μg/mL] and Cmax (600-800 ng/ml) (see FIG. 1).

Microscopic findings of similar nature and severity were recorded at most subcutaneous injection sites in groups 1-9. The microscopic reactions mostly comprised minimal inflammatory cell infiltrates and/or epidermal crusting, which are findings that are consistent with this route of administration.

The main readout for histological adverse reactions was thrombus formation (local blood clotting), which was recorded at the injection site within group 5 only (in 1 out of 3 animals), at a greater severity than that seen at the control site. Despite the greater severity of thrombus formation relative to the control injection in this group, when measured on an absolute scale of 1-5 (with 1 indicating “minimal” and 5 indicating “severe”), the thrombus was graded a 2 (slight vs. minimal in the other treatment groups), meaning that it is of limited severity.

The thrombus formation is believed to be associated with the high FIX protein concentration administered in that group (21.0 mg/mL), as groups that were administered a lower protein concentration did not show comparable thrombus formation at the injection site, even where the lower protein concentration corresponded to a higher total protein dose (e.g., group 5 vs group 4), or to a higher activity concentration (e.g., group 5 vs group 8). No clear correlation could be observed between thrombus formation and activity or protein dose per kg or per spot (per injection site), injection volume, or activity concentration. These principles were confirmed in a rabbit model, see Example 2.

Thus, it seems that the concentration of the FIX protein at the subcutaneous injection site is a major impacting factor on local tolerability. This was considered a surprising finding, suggesting that local tolerability is at least in part determined by the concentration of FIX molecules administered, rather than the FIX activity (either total or relative to volume). The findings also indicate that high-activity FIX variants are particularly suitable for subcutaneous administration of FIX because a therapeutically effective amount of FIX activity can be delivered while ensuring that the FIX protein concentration is sufficiently low to minimise local tolerability issues such as thrombus formation.

### Example 2

### Subcutaneously Administered Factor IX Wild-Type and High-Activity Variant in the Rabbit Model

Additional studies in rabbits were consistent with the results obtained in the pig model described in Example 1, i.e., that the concentration of the FIX protein at the subcutaneous injection site is a major impacting factor on local tolerability. However, while the data in the rabbit model provided a proof-of-principle, the absolute values cannot necessarily readily be applied to human subcutaneous use because the rabbit is a hypersensitive skin model (whereas porcine skin is physiologically very similar to human skin, as noted above), and because the systemic exposure of FIX following subcutaneous administration to rabbit is assumed to be ˜10-fold higher compared to the pig model (assuming that the bioavailability is the same and an about 10-fold difference in body weight).

Three groups of rabbits were tested as indicated in Table 3 below. Group 1 received a lower FIX activity dose with the aim to show no or only little effects: 1120 IU of R338L FIX as defined in SEQ ID NO: 10 (diluted in 20 mM Hepes, 100 mM isotonic saline, 10 mmol CaCl₂), pH 6.2) was administered in a volume of 1.6 mL (700 IU/mL FIX clotting activity with about 0.53 mL/kg, i.e. 1.6 mL per rabbit or 371 IU/kg) to the left hand side of the rabbit to n=3 female rabbits. Groups 2 and 3 received a comparable FIX activity dose and activity concentration. However, in light of the higher specific activity of FIX R338L, it was administered at a lower protein concentration (group 2). In group 2, 2000 IU of R338L FIX as defined in SEQ ID NO: 10 (diluted in 20 mM Hepes, 100 mM isotonic saline, 10 mmol CaCl₂), PH 6.2) was administered in a volume of 1.0 mL (2000 IU/mL FIX clotting activity with about 0.33 ml/kg, i.e. 1.0 mL per rabbit or 667 IU/kg) to the left hand side of the rabbit to n=3 female rabbits. In group 3, 2051 IU of WT FIX as defined in SEQ ID NO: 9 (diluted in 20 mM Hepes, 100 mM isotonic saline, 10 mM CaCl₂), pH 6.2) was administered in a volume of 1.0 mL (2051 IU/mL FIX clotting activity with about 0.33 mL/kg, i.e. 1.0 mL per rabbit or 684 IU/kg) to the left hand side of the rabbit to n=3 female rabbits.

TABLE 3

| | | Specific | Activity | Protein | | Activity | Protein | | | | | | |
|---|---|---|---|---|---|---|---|---|---|---|---|---|---|
| | | activity | dose per | dose per | | dose per | dose | | | Activity | | FIX protein | |
| Group | Protein | [IU/mg] | kg | kg | | spot | per spot | | Volume | concentration | | concentration | |
| 1 | HA | 338 | 371 IU/kg | 472 | μg/kg | 1120 IU | 1.4 | mg | 1.6 mL | 700 | IU/mL | 0.9 | mg/mL |
| 2 | HA | 338 | 667 IU/kg | 848 | μg/kg | 2000 IU | 2.5 | mg | 1.0 mL | 2000 | IU/mL | 2.5 | mg/mL |
| 3 | WT | 44 | 684 IU/kg | 6711 | μg/kg | 2051 IU | 20.1 | mg | 1.0 mL | 2051 | IU/mL | 20.1 | mg/mL |

“WT” = wild-type Factor IX;
“HA” = high-activity Factor IX

Local tolerability was recorded, including haemorrhage, thrombus, necrosis, inflammation of the epidermis, dermis, vessel, and/or muscle at the injection sites.

### Results

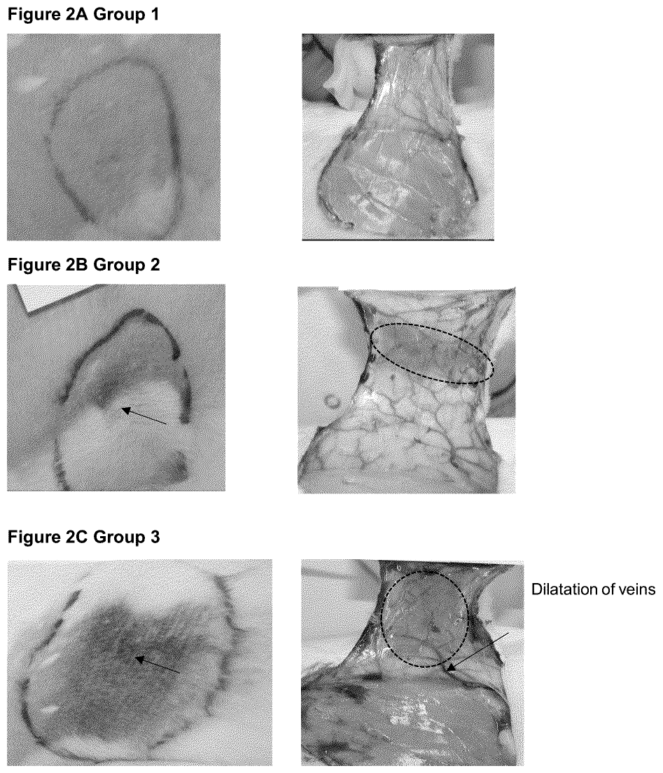

Findings in group 3 were generally of greater incidence and/or severity than those in group 2 (or group 1). At comparable activity dose, activity concentration and volume, group 2 (which received FIX at a lower protein concentration) was better tolerated than in group 3. Exemplary photographs are shown in FIG. 2 (A: group 1; B: group 2; C: group 3).

Microscopic findings were noted at most control subcutaneous injection sites after administration of vehicle and comprised minimal inflammatory cell infiltrates, minimal granuloma, minimal inflammation and/or epidermal crusting.

Microscopic findings were noted at the injection site after administration of ‘Padua’ mutant occurred in group 1 in two animals and comprised minimal inflammation of the epidermis/dermis and subcutis and slight inflammation of the underlying muscle. In addition, there was minimal vascular/perivascular inflammation and slight thrombus formation. In group 2, slight thrombus, and minimal to moderate inflammation of the dermis and vessel were recorded for 2 animals. In addition, slight haemorrhage and minimal inflammation of the muscle was recorded for one animal. Finally, in group 3, following WT FIX (in the form of Idelvion®) injection, minimal to moderate haemorrhage, thrombus and/or inflammation of the epidermis, dermis, vessel, and/or muscle, were recorded. In addition, minimal focal necrosis was recorded for the epidermis for one animal. Taken together, a gradual increase of histologic findings was observed from group 1 to 2 and even more severe findings, including necrosis, were observed in group 3, i.e. the group that received the highest FIX protein concentration.

### Methods

### Determination of FIX Activity

FIX activity was determined as clotting or coagulation activity (FIX:C) with a one-stage clotting assay, using commercially available aPTT reagents. Briefly, a mixture of the pre-diluted test sample and FIX-deficient plasma is incubated with a defined amount of activator PathromtinSL™ (Siemens Healthineers, Marburg, Germany). The reaction is started by adding calcium chloride, which leads to activation of the intrinsic coagulation pathway. A FIX deficiency of the intrinsic coagulation pathway prolongs activated partial thromboplastin time (aPTT) in a concentration-dependent manner. An internal substandard calibrated against the WHO International FIX concentrate Standard was used as a reference. The measured coagulation times are converted into IU/mL using a reference curve prepared from standard human plasma (SHPL) calibrated from the manufacturer against the World Health Organization (WHO) standard (contains International Blood Coagulation Factors II, VII, IX, X, Human, Plasma) for FIX.

### Determination of FIX Antigen

FIX antigen (FIX:Ag) was determined by an ELISA according to standard protocols known to those skilled in the art. Briefly, microtiter plates were incubated with 100 μL per well of the capture antibody (Paired antibodies for FIX ELISA (CL20041K), Cedarlane, but other sources of appropriate antibodies may also be applied) overnight at ambient temperature. After washing plates three times with washing buffer B (Sigma T9039) each well was incubated with 200 μL blocking buffer C (Sigma P3688) for one hour at ambient temperature. After another three wash steps with buffer B, serial dilutions of the test sample in buffer B as well as serial dilutions of a substandard (SHP) in buffer B (volumes per well: 100 μL) were incubated for 90 min at ambient temperature. After three wash steps with buffer B, 100 ml of a 1:200 dilution of the detection antibody (Paired antibodies for FIX ELISA, peroxidase labelled, Cedarlane) in buffer B, were added to each well and incubated for another 90 min at ambient temperature. After three wash steps with buffer B, 100 μL of substrate solution (TMB, Siemens Healthcare, OUVF) were added per well and incubated for 30 minutes at ambient temperature in the dark. Addition of 100 μL undiluted stop solution (Siemens Healthcare, OSFA) prepared the samples for reading in a suitable microplate reader at 450 nm wavelength. Concentrations of test samples were then calculated using the standard curve with standard human plasma as reference.

### Determination of FIX Protein Amount Via OD Measurement

Protein content by OD was determined using the Little Lunatic (Unchained Labs, Pleasanton, CA 94566) instrument. For the measurement, 2 μL of the sample and x1 the corresponding buffer were added to the Little Lunatic chip. If the extinction coefficient (0.9) is provided to the instrument, it gives the final protein concentration of the rFIX sample.

To correct the protein mass for the albumin weight, the following assumptions were taken: molecular mass was calculated from the FASTA-sequence of the molecules (FIX=52 kDa, albumin=69 kDa, albumin-fused FIX=121 kDa). On this basis, the mass of the FIX portion of the albumin-fusion protein is 43% of the total protein mass.

It will be understood that the invention has been described by way of example only and modifications may be made whilst remaining within the scope and spirit of the invention.

### REFERENCES

[1] Simioni et al. (2009) N Engl J Med. Oct 22; 361(17):1671-1675
[2] WO 2020/187969 A1
[3] Perot et al. (2015) Thromb. Res. 135, 1017-1024
[4] Quade-Lyssy et al. (2014) J. Thromb. Haemost. 12, 932-942
[5] Cheung et al. (1992) J. Biol. Chem. 267, 20529-20531
[6] Lin et al. (2010) J. Thromb. Haemost. 8, 1773-1783
[7] Sichler et al. (2003) J. Biol. Chem. 278, 4121-4126
[8] Milanov et al. (2012) Blood 119, 602-611
[9] Misenheimer et al. (2007) Biochemistry 46, 7886-7895
[10] S.-B. Hong et al. (2016) Am. Soc. Hematology, abstract
[11] Kao et al. (2013) Thromb. Haemost. 110, 244-256
[12] Beattie & Dugaiczyk (1982) Gene 20:415-422
[13] WO 2005/024044
[14] Lichenstein et al. (1994) J. Biol. Chem. 269:18149-18154
[15] Cooke & David (1985) J. Clin. Invest. 76:2420-2424
[16] WO 2005/001025
[17] Powell et al. (2013) N. Engl. J. Med., 369:2313-2323
[18] Peters et al. (2010) Blood 115:2057-2064
[19] Shapiro et al. (2012) Blood 119:666-672
[20] Fares et al. (1992) Proc Natl Acad Sci USA 15; 89(10):4304-4308
[21] Calo et al. (2015) Precision Medicine, 2, e989
[22] WO 2011/004361
[23] Schellenberger et al. (2009) Nature Biotechnology 27, 1186-1190
[24] WO 2017/024060
[25] WO 2012/006624
[26] WO 2015/106052
[27] Collins et al. (2014) Blood 124:3880-3886
[28] WO 2006/127896
[29] WO 2005/055950
[30] DeFrees et al. (2006) Glycobiology 16(9):833-843
[31] Gennaro (2000) Remington: The Science and Practice of Pharmacy. 20th edition, ISBN: 0683306472.
[32] Summerfield et al. (2015) Mol Immun 66:14-21.

1-13. (canceled)

14. A pharmaceutical composition suitable for subcutaneous administration, wherein the pharmaceutical composition comprises a high-activity Factor IX (FIX) variant polypeptide connected to a half-life enhancing portion, wherein the FIX variant polypeptide has a specific molar activity that is at least 2 times higher than the specific molar activity of a FIX polypeptide containing wild-type FIX, and wherein the pharmaceutical composition comprises the FIX variant polypeptide at a FIX protein concentration of no more than 20 mg/mL and in a total volume of no more than 5 mL per unit dose.

15. (canceled)

16. A method of treating or preventing a bleeding disorder in a subject in need thereof, comprising subcutaneously administering to the subject a high-activity Factor IX (FIX) variant polypeptide connected to a half-life enhancing portion at a FIX protein concentration of no more than 20 mg/mL and a total volume of no more than 5 mL per administration site, wherein the FIX variant polypeptide has a specific molar activity that is at least 2 times higher than the specific molar activity of a FIX polypeptide containing wild-type FIX.

17. The method of claim 16, wherein the FIX variant polypeptide is administered at a FIX protein concentration of no more than 19 mg/mL per administration site.

18. The method of claim 16, wherein the FIX variant polypeptides is administered in a total volume of no more than 4.5 mL per administration site.

19. The method of claim 18, wherein the FIX variant polypeptide is administered in a total volume of no more than 1 mL per administration site.

20. The method of claim 19, wherein the FIX variant polypeptide is administered at a FIX protein concentration of no more than 15.8 mg/mL.

21. The method of claim 20, wherein the FIX variant polypeptide is administered in a total volume of no more than 0.5 mL per administration site.

22. The method of claim 16, wherein the FIX variant polypeptide has a specific molar activity that is at least 2.5 times higher than the specific molar activity of a FIX polypeptide containing wild-type FIX.

23. The method of claim 22, wherein the FIX variant polypeptide has a specific molar activity that is at least 3 times higher than the specific molar activity of a FIX polypeptide containing wild-type FIX.

24. The method of claim 16, wherein the FIX variant polypeptide comprises a leucine at a position corresponding to position 338 of wild-type Factor IX as defined in SEQ ID NO: 1.

25. The method of claim 16, wherein the FIX variant polypeptide comprises an amino acid sequence having at least 70% sequence identity to SEQ ID NO: 1.

26. The method of claim 16, wherein the FIX variant polypeptide comprises an amino acid sequence as defined in SEQ ID NO: 8.

27. The method of claim 16, wherein the half-life enhancing portion is albumin including variants and derivatives thereof, or a polypeptide of the albumin family including variants and derivatives thereof.

28. The method of claim 27, wherein the FIX variant polypeptide comprises an amino acid sequence as defined in SEQ ID NO: 10.

29. The method of claim 16, wherein the bleeding disorder is hemophilia B.


SEQUENCE LISTING (20 sequences)

SEQ ID NO: 1 - protein, 415 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 2 - DNA, 1251 bp, Homo sapiens
atgtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 3 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 4 - DNA, 1386 bp, Homo sapiens
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 5 - protein, 145 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTR

SEQ ID NO: 6 - protein, 235 aa, Homo sapiens
VVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 7 - protein, 415 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAEAVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 8 - protein, 415 aa, Artificial Sequence
  R338L FIX variant polypeptide
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT

SEQ ID NO: 9 - protein, 1018 aa, Artificial Sequence
  FIX (wild-type) albumin fusion protein
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTPVSQTSKLTRAETVFPDVDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 10 - protein, 1018 aa, Artificial Sequence
  R338L FIX variant polypeptide albumin fusion
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLTPVSQTSKLTRAETVFPDVDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 11 - protein, 18 aa, Artificial Sequence
  Linker
PVSQTSKLTRAETVFPDV

SEQ ID NO: 12 - protein, 585 aa, Homo sapiens [sequence omitted]

SEQ ID NO: 13 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 10 aa, Artificial Sequence
  CTP sequence
SSSSKAPPPS

SEQ ID NO: 16 - protein, 32 aa, Artificial Sequence
  CTP sequence
DPRFQDSSSSKAPPPSLPSPSRLPGPSDTPIL

SEQ ID NO: 17 - protein, 28 aa, Artificial Sequence
  CTP sequence
SSSSKAPPPSLPSPSRLPGPSDTPILPQ

SEQ ID NO: 18 - protein, 78 aa, Artificial Sequence
  XTEN artificial sequence
GAPTSESATPESGPGSEPATSGSETPGTSESATPESGPGSEPATSGSETPGTSESATPESGPGTSTEPSEGSAPGASS

SEQ ID NO: 19 - protein, 42 aa, Artificial Sequence
  XTEN artificial sequence
GAPGSPAGSPTSTEEGTSESATPESGPGSEPATSGSETPASS

SEQ ID NO: 20 - protein, 7 aa, Artificial Sequence
  Linker
GGGGGGV